(19) United States
(12) Patent Application Publication
(10) Pub. No.: US 2007/0228184 A1
(43) Pub. Date: Oct. 4, 2007

(54) Aromatic container

(76) Inventor: Yu-Gang Chen, Yongkang City (TW)

Correspondence Address: Far East College, P. O. Box No. 6-57, Junghe, Taipei, 235, TW

(21) Appl. No.: 11/395,203

(22) Filed: Apr. 3, 2006

(51) Int. Cl. A61L 9/04

(52) U.S. Cl. 239/36; 239/58

(57) ABSTRACT

An aromatic container includes a first part which is provided with a first hollow part and a first assembly part; a second part which is provided with a second hollow part and a second assembly part; an assembly part which assembles the first part and the second part, which can be shifted relative to each other, into a closed space; an additive member which is fixed at the first part or the second part; and aromatics which are emplaced in the aforementioned closed space. The aromatic container can be worn on a human body with the additive member, and can be freely opened and closed by a relative shifting between the aforementioned first part and the second part.

### BACKGROUND OF THE INVENTION

(a) Field of the Invention

The present invention relates to an aromatic container and more particular to an aromatic container which can be worn as well as freely opened and closed.

(b) Description of the Prior Art

In using a conventional aromatic container, a package of aromatics is removed and then the aromatics are emplaced in the container so as to gradually scatter a fragrance of the aromatics to an ambient environment from apertures on a cover. As the cover is not provided with any enclosure device, the aromatics in the container will not be able to be sealed in the container after removing the package, and can only gradually dissipate in an air. Furthermore, the aromatic container can be only fixed at a certain place and cannot be carried personally.

On the other hand, a perfume which is sprayed on a human body is provided with a good effect of fragrance; however, the fragrance is scattering continuously after being sprayed on the human body, which will enable a person who is sensitive to the fragrance to be unbearable if staying in a closed space such as an elevator. In addition, the fragrance of perfume sprayed on the human body can only persist for a period of time, and is easy to quickly disappear especially when the person is sweating.

### SUMMARY OF THE INVENTION

Accordingly, the primary object of present invention is to solve problems that an aromatic container cannot be worn personally and a time of scattering its fragrance cannot be controlled freely.

The present invention includes a first part which is provided with a first hollow part and a first assembly part, a second part which is provided with a second hollow part and a second assembly part, an assembly part which assembles the aforementioned first part and the second part into a closed space wherein the first and the second parts can be shifted relative to each other, an additive member which is fixed at the first part or the second part, aromatics which are emplaced in the aforementioned closed space. The first hollow part and the second hollow part are staggered or overlapped with each other by a relative shifting between the aforementioned first part and the second part.

An exterior surface of the aforementioned closed space is provided with a decoration unit which is fixed at the first part or the second part.

The aforementioned additive member is a component which can be worn.

The aforementioned additive member is a component having perforations.

The present invention is provided with following advantages:
1. When a user needs to scatter a fragrance with the present invention, he or she can move the first part and the second part to enable the first hollow part to be overlapped with the second hollow part, thereby allowing the aromatics in the container to scatter the fragrance.
2. If the user does not need to scatter the fragrance, he or she can move the first part and the second part to enable the first hollow part to be staggered with the second hollow part, thereby prohibiting the aromatics from scattering the fragrance out of the container.
3. The additive member can be a clip, a necklace, an ear ring, a hairpin, an ornament of cellular phone, a scarf pin, a tie clip, a headband, a wrist band, and a key ring, which is easy to be carried without occupying a space.
4. An exterior surface of the closed space of aromatic container can be attached with any decoration member, which is beauty to wear without being abrupt.
5. The aromatics of aromatic container can be replaced and a fragrance can be changed according to a situation or mood.
6. The aromatics only scatter the fragrance when necessary, which is more economic in consuming the aromatics.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows an exploded view of an embodiment of the present invention including aromatics.

FIG. 2 shows an overall cutaway view of an embodiment of the present invention including aromatics, wherein a first hollow space is completely overlapped with a second hollow part.

FIG. 3 shows an overall cutaway view of an embodiment of the present invention including aromatics, wherein a first hollow space is partially overlapped with a second hollow part.

FIG. 4 shows an overall cutaway view of an embodiment of the present invention including aromatics, wherein a first hollow space is staggered with a second hollow part.

FIG. 5 shows a schematic view of the present invention being worn on a human body.

FIG. 6 shows a cutaway view of a first part and a second part shown in an equivalent configuration of the present invention including aromatics.

### DETAILED DESCRIPTION OF THE PREFERRED EMBODIMENTS

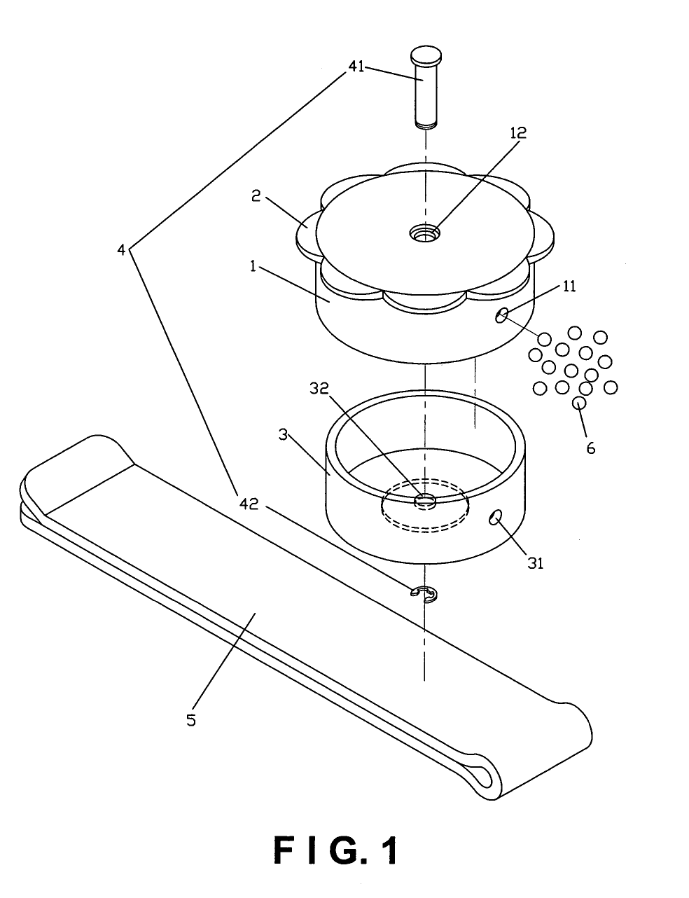

Referring to FIG. 1, it shows an embodiment of the present invention including aromatics. A first part 1 is a casing having a concaved space, and a surface of which is provided with a decoration unit 2 which is a piece of ornament having an appearance of flower petal. The first part 1 is provided with a first hollow part 11 which is a through-hole, and is also provided with a first assembly part 12 which is a circular hole.

A second part 3 is a casing having a concaved space and is provided with a second hollow part 31 which is a through-hole. The second part 3 is also provided with a second assembly part 32 which is a circular hole.

An assembly part 4 includes a bolt 41 and a C-shape hook 42.

An additive member 5 is a hairpin which is fixed (such as pasted) on the aforementioned second part 3.

Aromatics 6 contain an ingredient of spice and are grains which can be gradually vaporized.

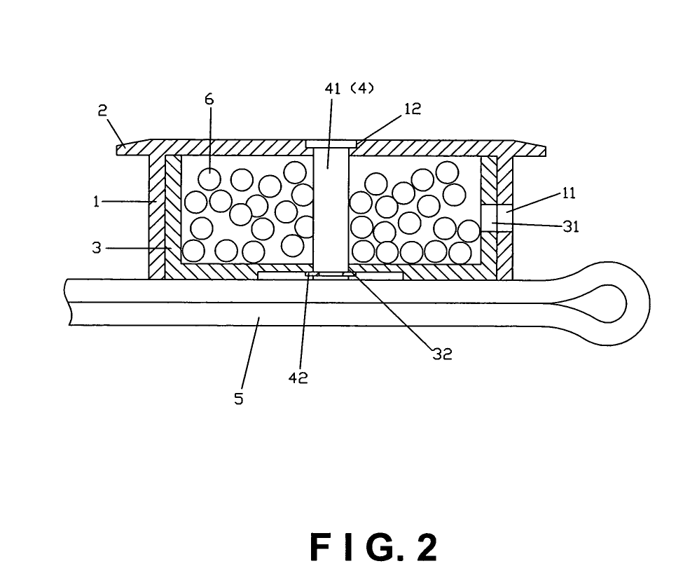

Referring to FIG. 1 and FIG. 2, the bolt 41 of assembly part 4 is transfixed into the first assembly part 12 of first part 1 and the second assembly part 32 of second part 3, and is locked by the C-shape hook 42 to assemble the first part 1 and the second part 3, which can be shifted relative to each other, into a closed space. The aromatics 6 are emplaced in the aforementioned closed space. The first hollow part 11 and the second hollow part 31 are completely overlapped with each other by rotating the first part 11 relative to the second part 31, thereby forming an entrance for filling the aromatics 6 into the closed space.

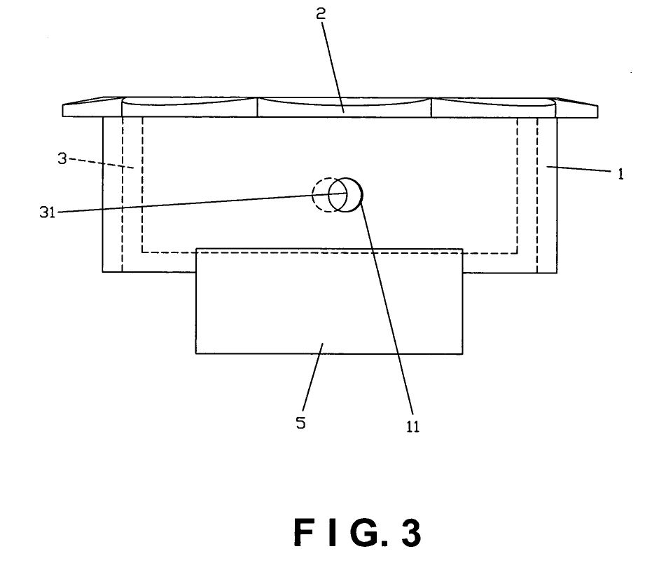

Referring to FIG. 3, the first hollow part 11 and the second hollow part 31 are partially overlapped with each other by rotating the first part 11 relative to the second part 31, thereby forming an opening to scatter the fragrance without dropping out the aromatics 6 (not shown in the drawing), and enabling the aromatics 6 to scatter the fragrance from the closed space.

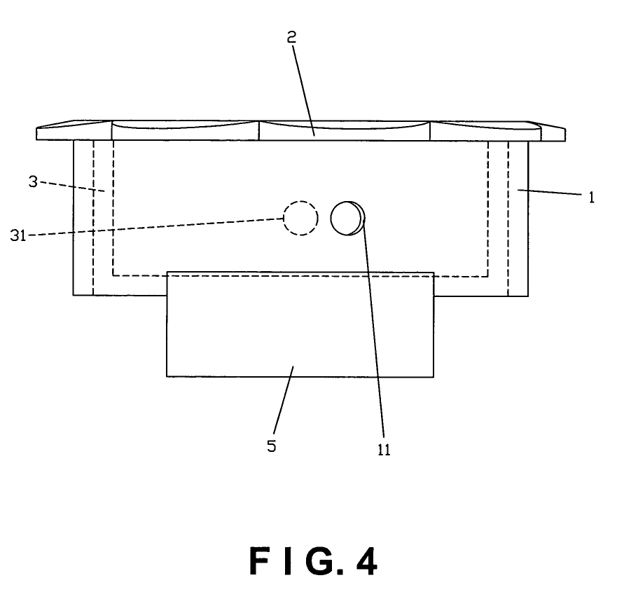

Referring to FIG. 4, the first hollow part 11 and the second hollow part 31 are staggered with each other by rotating the first part 11 relative to the second part 31, thereby completely sealing the closed space to prohibit the aromatics 6 (not shown in the drawing) from scattering the fragrance out of the closed space.

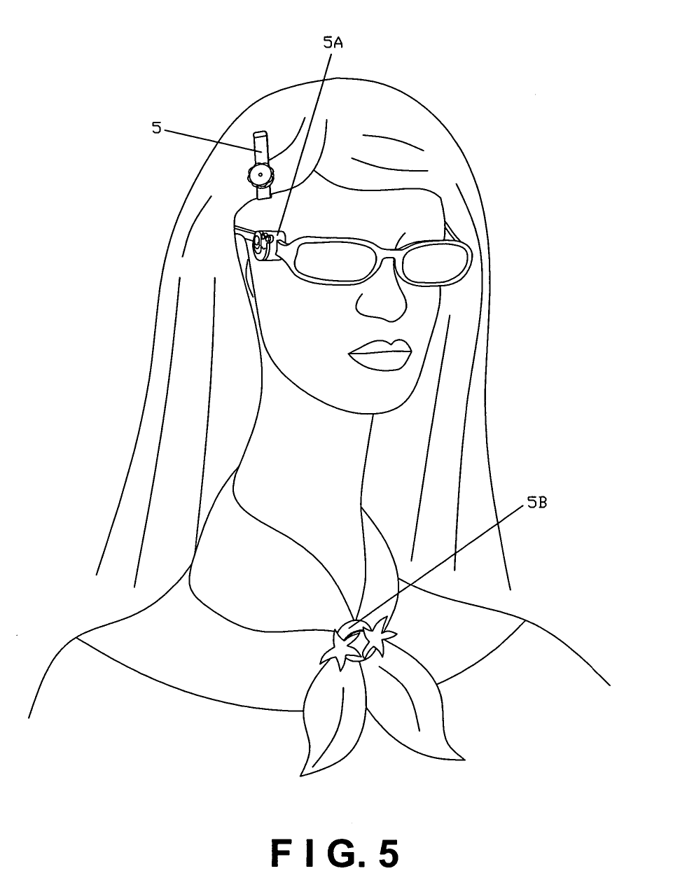

Referring to FIG. 5, the additive member 5 is a hairpin being worn on a human body to manifest a hairpin with an ornament, which can completely hide the aforementioned closed space without having a vision of abruptness of wearing an aromatic container. An additive member 5A is a clip which can be worn on a foot of frame of glasses; whereas an additive member 5B is a bind ring which is used to bind a scarf.

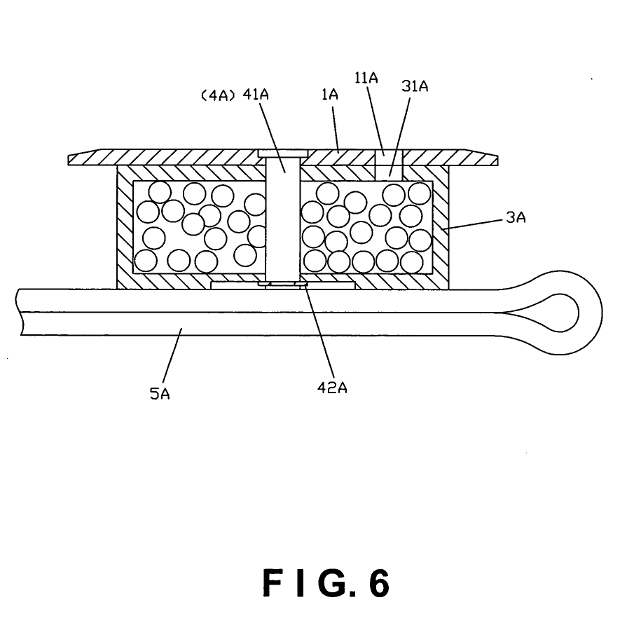

The first part 1 and the second part 3 are components used for assembling into the aforementioned closed space. A plurality of equivalent changes can be made to shapes of the first part 1 and the second part 3. Referring to FIG. 6, a first part 1A is a plate and is provided with a first hollow part 11A, a second part 3A is a closed box and is provided with a second hollow part 31A, and an additive member 5A is fixed at the second part 3A. By a bolt 41A and a C-shape hook 42A of an assembly part 4A, the first part 1A and the second part 3A can be assembled into a configuration wherein they can be shifted relative to each other, which is an equivalent configuration to the present invention.

An aforementioned relative shifting between the first part 1 and the second part 3 includes a rotation and a slide.

The aforementioned assembly part 4 can also be a type of mutually corresponding objects, such as a shaft hole and a shaft, a tenon and a tenon hole, which are directly formed on the first part 1 and the second part 3 respectively, to assemble the first part 1 and the second part 3 by sheathing.

The decoration unit 2 of present invention can be integrally formed on the first part 1 or the second part 3, or can also be assembled on the first part 1 or the second part 3 by packaging, sticking, or enchasing.

The aromatics 6 of present invention can be a solid object, or a liquid object in association with a sponge.

The additive member 5 can be a component with a circular hole for providing for transfixing with a rope to form a hanging configuration, such as a type of necklace.

The present invention can be provided with additive members of different styles, including a clip, a necklace, an ear ring, an ornament of cellular phone, a tie clip, a headband, a wrist band, and a key ring, which can be carried.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. An aromatic container comprising a first part which is provided with a first hollow part, a second part which is provided with a second hollow part, and an additive member which is fixed at the first part or the second part; the first part and the second part being able to be shifted relative to each other for assembling into a closed space, wherein the relative shifting between the first part and the second part enables the first hollow part to be staggered or overlapped with the second hollow part.

2. The aromatic container according to claim 1, wherein an exterior surface of the closed space is provided with a decoration unit which is fixed at the first part or the second part.

3. The aromatic container according to claim 1, wherein the additive member is a component which can be worn.

4. The aromatic container according to claim 1, wherein the additive member is a component having perforations.

5. An aromatic container comprising a first part which is provided with a first hollow part and a first assembly part; a second part which is provided with a second hollow part and a second assembly part; an assembly part which assembles the aforementioned first part and the second part, which can be shifted relative to each other, into a closed space; and an additive member which is fixed at the first part or the second part; the first hollow part being staggered or overlapped with the second hollow part by a relative shifting between the aforementioned first part and the second part.

6. The aromatic container according to claim 5, wherein an external surface of the closed space is provided with a decoration unit which is fixed at the first part or the second part.

7. The aromatic container according to claim 5, wherein the additive member is a component which can be worn.

8. The aromatic container according to claim 5, wherein the additive member is a component having perforations.

9. An aromatic container comprising a first part which is provided with a first hollow part and a first assembly part; a second part which is provided with a second hollow part and a second assembly part; an assembly part which assembles the aforementioned first part and the second part, which can be shifted relative to each other, into a closed space; an additive member which is fixed at the first part or the second part; and
aromatics which are emplaced in the aforementioned closed space; the first hollow part being staggered or overlapped with the second hollow part by a relative shifting between the aforementioned first part and the second part.

10. The aromatic container according to claim 9, wherein an external surface of the closed space is provided with a decoration unit which is fixed at the first part or the second part.

11. The aromatic container according to claim 9, wherein the additive member is a component which can be worn.

12. The aromatic container according to claim 9, wherein the additive member is a component having perforations.
